(19) United States
(12) Patent Application Publication
(10) Pub. No.: US 2007/0225225 A1
(43) Pub. Date: Sep. 27, 2007

(54) USE OF NATRIURETIC PEPTIDE RECEPTOR ANTAGONISTS TO TREAT OCULAR, OTIC AND NASAL EDEMETOUS CONDITIONS

(76) Inventors: Najam A. Sharif, Arlington, TX (US); Parvaneh Katoli, Arlington, TX (US)

Correspondence Address: ALCON, IP LEGAL, TB4-8, 6201 SOUTH FREEWAY, FORT WORTH, TX, 76134, US

(21) Appl. No.: 11/679,991

(22) Filed: Feb. 28, 2007

Related U.S. Application Data
(60) Provisional application No. 60/777,686, filed on Feb. 28, 2006.

(51) Int. Cl. A61K 38/17; A61K 31/404

(52) U.S. Cl. 514/12; 514/418

(57) ABSTRACT

Methods and compositions to treat edematous ocular, otic and nasal conditions are described.

The present application claims the benefit of U.S. Provisional Patent Application Ser. No. 60/777,686 filed Feb. 28, 2006.

### BACKGROUND OF THE INVENTION

1. Field of the Invention

The present invention relates to the field of treatment of ocular, otic and/or nasal edematous conditions. More specifically, the present invention relates to the use of antagonists of natriuretic peptide receptors to treat such conditions.

2. Description of the Related Art

Ocular surface inflammation caused by chemical, mechanical and/or environmental factors can lead to edematous conditions of the cornea and/or conjunctiva that can seriously distort vision and thus impact quality of life (Hess and Carney, Vision through an abnormal cornea: a pilot study of the relationship between visual loss from corneal distortion, corneal edema, ketacoconus, and some allied corneal pathology. Invest. Opthalmol. Vis. Sci. 18: 476-483, 1979; Herse, Recovery from contact lens-induced edema is prolonged in the diabetic rabbit cornea. Optom. Vis. Sci. 67: 466-470, 1990). In cornea pseudoguttata, thickening of the Decemet's membrane and subsequent disruptions of the lesion causes edema in the corneal endothelia cell layer and disrupts vision similarly to iritis and corneal inflammation (Krachmer et al. Cornea pseudoguttata: a clinical and histopathological description of endothelial cell edema. Arch. Opthalmol. 99: 1377-1381, 1981). Corticosteroids can be used to reduce the swelling but this can lead to a serious side-effect of raising the intraocular pressure (IOP) which over time can cause blindness (Melberg and Olk. Corticosteroid-induced ocular hypertension in treatment of aphakic or pseudoaphakic cystoid macular edema. Ophthalomol. 100: 164-167, 1993).

In the back of the eye, edematous conditions can be caused by such disorders as migraine (Victor and Welch. Bilateral retinal hemorrhages and disk edema in migraine. Am. J. Opthalmol. 84: 555-558, 1977), hypotonia (Turut et al. Fluorographic aslects of papillo-retinal edema due to hyptonia. Bull. Soc. Opthalmol. France. 74: 486-492, 1974), cataract surgery (Moses. Cystoid macular edema and retinal detachment following cataract surgery. J. Am. Intraocul. Implant Soc. 5: 326-329, 1979), retinal vein occlusion (Gutman. Macular edema in branch retinal vein occlusion: prognosis and management. Trans. Sect. Opthalmol. Am. Acad. Opthalmol. Otolaryngol. 83: 488-495, 1977), sarcoidosis (Throne and Galetta. Disc edema and retinal periphlebitis as the intial mansifestation of sarcoidosis. Arch. Neurol. 55: 862-863, 1998), hypotony maculopathy (Kokame et al. Serous retinal detachment and cystoid macular edema in hypotony maculopathy. Am. J. Opthalmol. 131: 384-386, 2001) and diabetic retinopathy (Strom et al. Effect of ruboxistaurin on blood-retinal barrier permeability in relation to severity of leakage in diabetic macular edema. Invest. Opthalmol. Vis. Sci. 46: 3855-3858, 2005). Retinal edema has been treated with corticosteroids (Cekic et al. Intravitreal triamcinolone treatment for macular edema associated with central vein occulusion and hemiretinal vein occlusion. Retina. 25: 846-850, 2005), antibodies (Rosenfeld et al. Optical coherence tomography findings after an intravitreal injection of bevacizumab (avastatin) for macular edema from retinal vein occlusion. Ophthalmic Surg. Lasers Imaging. 36: 336-339, 2005), laser treatment (Esrick et al. Multiple laser treatments for macular edmema attributable to branch vein occlusion. Am. J. Opthalmol. 139: 653-657, 2005), protein kinas C inhibitors (Strom et al. Effect of ruboxistaurin on blood-retinal barrier permeability in relation to severity of leakage in diabetic macular edema. Invest. Opthalmol. Vis. Sci. 46: 3855-3858, 2005), cyclooxygenase inhibitors (Kapin et al. Inflammation-mediated retinal edema in the rabbit is inhibited by topical nepafenac. Inflammation. 27: 281-289, 2003) and hyperbaric oxygen (Kiryu and Ogura. Hyperbaric oxygen treatment for macular edema in retinal vein occlusion: relation to severity of retinal leakage. Opthalmologica, 210: 168-170, 1996). Unfortunately, many of the treatments above are plagued by serious side-effects such as collateral damage after laser treatment, steroid-induced cataract and development of glaucoma, that limit their utility (Marmor. Mechanisms of fluid accumulation in retinal edema. Doc. Opthalmol. 97: 239-249, 1999). Thus, new pharmacological agents are needed to treat retinal and corneal edematous conditions of the eye.

Otic edema can cause serious hearing loss (Ravicz. Mechanism of hearing loss resulting from middle-ear fluid. Hearing Res. 195: 103-130, 2004; Kafer, Proportioned dwarfism combined with ocular myopathy, recurring corneal edema, tapetoretinal degeneration, hearing loss of the inner ear and diabetis mellitus. Ber. Zusammenkunft. Dutch. Opthalmol. Ges. 73: 618-620, 1975; Simmons. Fluid dynamics in sudden sensorineural hearing loss. Otolaryngol. Clin. North Am. 11: 55-61, 1978; Priner et al. The neonate has a temporary conductive hearing loss due to fluid in the middle ear. Audiol. Neurootol. 8: 100-110, 2003). Apart from corticosteroids, new pharmacological agents are needed to treat such fluid accumulation in the ear to limit hearing loss.

Nasal mucosal edema can result from irritant chemicals and/or pathogenic agents in the air and such edema can complicate treatment for pulmonary diseases (Ranga and Ackerman. Edema of the nasal mucosa complicating treatment of respiratory distress syndrome. Am. J. Dis. Child. 132: 96, 1978; Berdal. Serological investigations on the edema fluid from nasal polyps; a preliminary report. J. Allergy 23: 11-14, 1952; Grudemo. The effects of saline-induced edema in the human nasal mucosa on laser Doppler-flowmetry. Rhinology 37: 1040107, 1999). Once again, corticosteroids are used to reduce such nasal/pulmonary edema but the long term use of such agents is not recommended and can be complicated further by infections such that new therapeutics agents are needed to address such nasal/pulmonary edematous disorders (Ranga and Ackerman. Edema of the nasal mucosa complicating treatment of respiratory distress syndrome. Am. J. Dis. Child. 132: 96, 1978; Berdal. Serological investigations on the edema fluid from nasal polyps; a preliminary report. J. Allergy 23: 11-14, 1952; Grudemo. The effects of saline-induced edema in the human nasal mucosa on laser Doppler-flowmetry. Rhinology 37: 1040107, 1999.

The natriuretic peptides are a family of related peptides with high sequence homology particularly within a common 17-amino acid disulfide ring structure. They are products of at least three distinct genes and they activate at least two types of receptors coupled positively to guanylate cyclase and thus elevate cGMP levels in cells (Anand-Srivastava and Trachte, Atrial natriuretic factor receptors and signal transduction mechanisms. Pharmacol. Rev. 45: 455-497, 1993). Atrial natriuretic peptide (ANP) receptors that are activated by ANP and brain natriuretic peptide (BNP) are termed type-A receptors, whereas the B-type receptors selectively bind the C-type natriuretic peptide (CNP). These peptides cause diuresis and natriuresis and hence modulate fluid homeostasis (Anand-Srivastava and Trachte, Atrial natriuretic factor receptors and signal transduction mechanisms. Pharmacol. Rev. 45: 455-497, 1993).

In terms of the eye, ANP and related peptides have been detected in the lacrimal gland (Lange et al., Localization of atrial natriuretic peptide/cardiodilatin (ANP/CDD)-immunoreactivity in the lacrimal gland of the domestic pig. Exp. Eye Res. 50: 313-316, 1990) and BNP-like immunoreactive nerves have been found in various ocular tissues including the porcine cornea (Yamamoto et al., Brain natriuretic peptide-immunoreactive nerves in the porcine eye. Neurosci. Lett. 122: 151-151, 1991). ANP and CNP synthesis, and CNP-binding sites, occur in the bovine corneal endothelium (Walkenbach et al. Atrial natriuretic peptide receptors on the corneal endothelium. Invest. Opthalmol. Vis. Sci. 34: 2538-2543, 1993; Sung et al., Coexistence of C-type natriuretic peptide and atrial natriuretic peptide systems in the bovine cornea. Invest. Opthalmol. Vis. Sci. 41: 2671-2677, 2000). Lastly, ANP was found to stimulate cGMP in rabbit corneal epithelial cells and ANP inhibited corneal wound healing induced by epidermal growth factor (Zhang et al. Effects of atrial natriuretic peptide and sodium nitroprusside on epidermal growth factor-stimulated wound repair in rabbit corneal epithelial cells. Curr. Eye Res. 21: 748-756, 2001). To-date only two non-peptide antagonists of the natriuretic peptide receptors have been described, namely HS-142-1 (Matsuda, Y. Design and utilization of natriuretic peptide antagonists. In: Contemporary Endocrinology: Natriuretic Peptides in Health and Disease. (Samson, W. K. and Levin, E. R.; Editors), Humana Press Inc., Totowa, N.J. (1997); Matsuda, Y and Morishita, Y. HS-142-1: a novel nonpeptide atrial natriuretic peptide antagonist of microbial origin. Cardiovasc. Drug Rev. 11: 45-59, 1993) and isatin (Medvedev et al. Interaction of isatin with type-A natriuretic peptide receptor: possible mechanism. Life Sci. 62: 2391-2398, 1998; Medvedev et al. Efficacy of isatin analogues as antagonists of rat brain heart natriuretic peptide receptors coupled to particulate guanylyl cyclase. Biochem. Pharmacol. 57: 913-915, 1999).

### SUMMARY OF THE INVENTION

The present invention overcomes these and other drawbacks of the prior art by providing a novel mechanism of action involving a peptidergic receptor-based system that is known to be involved in fluid regulation.

### BRIEF DESCRIPTION OF THE DRAWINGS

The following drawing forms part of the present specification and is included to further demonstrate certain aspects of the present invention. The invention may be better understood by reference to this drawing in combination with the detailed description of specific embodiments presented herein.

FIG. 1 illustrates the production of cGMP by natriuretic peptides in primary human corneal epithelial cells.

FIG. 2 illustrates the production of cGMP by natriuretic peptides in immortalized human corneal epithelial (CEPI-17-CL4) cells.

FIG. 3 illustrates the antagonism of CNP fragment-induced cGMP production by HS-142-1 in primary human corneal epithelial cells.

FIG. 4 illustrates the antagonism of CNP fragment-induced cGMP production by HS-142-1 in immortalized human corneal epithelial (CEPI-17-CL4) cells.

FIG. 5 illustrates the antagonism of CNP fragment-induced cGMP production by isatin in immortalized human corneal epithelial (CEPI-17-CL4) cells.

FIG. 6 illustrates the production of cGMP by natriuretic peptides in human conjunctival epithelial cells (Chang cells)

### DETAILED DESCRIPTION OF PREFERRED EMBODIMENTS

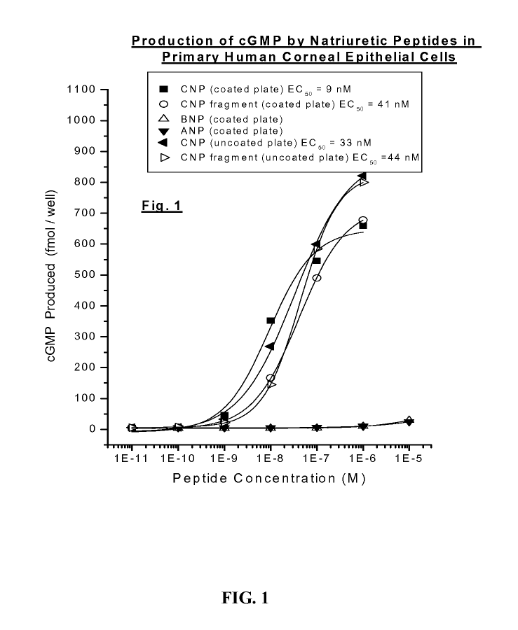

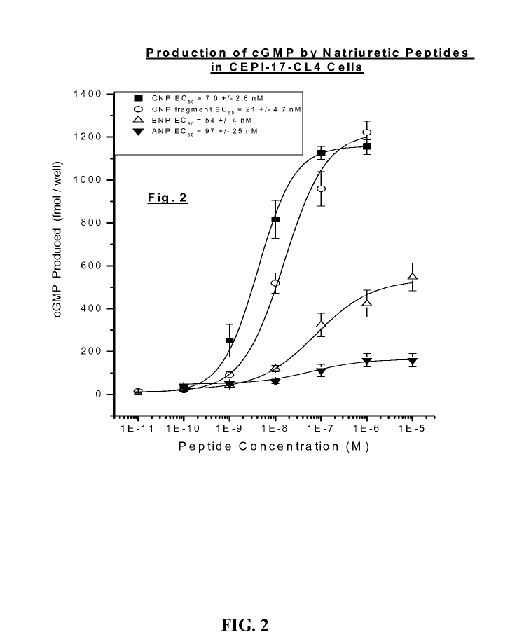

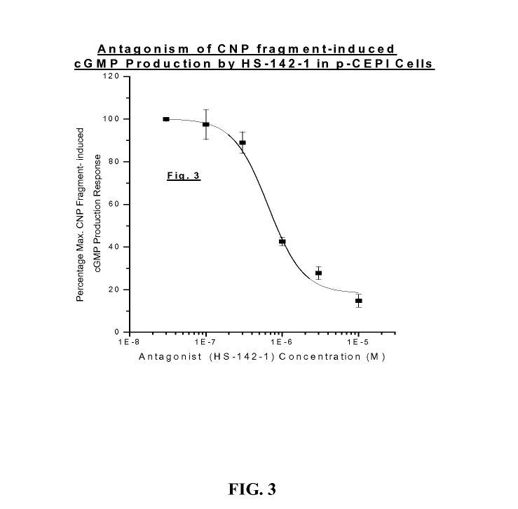

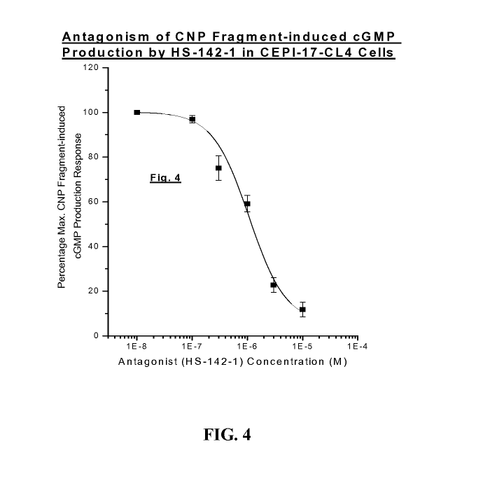

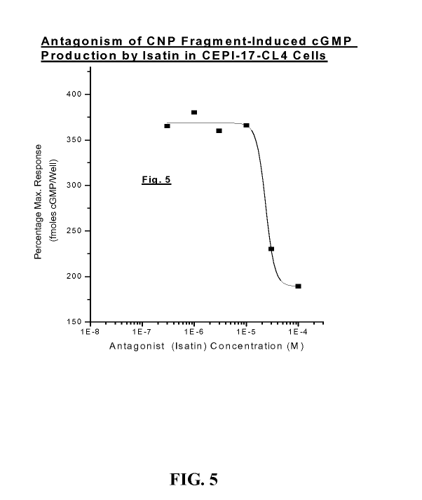

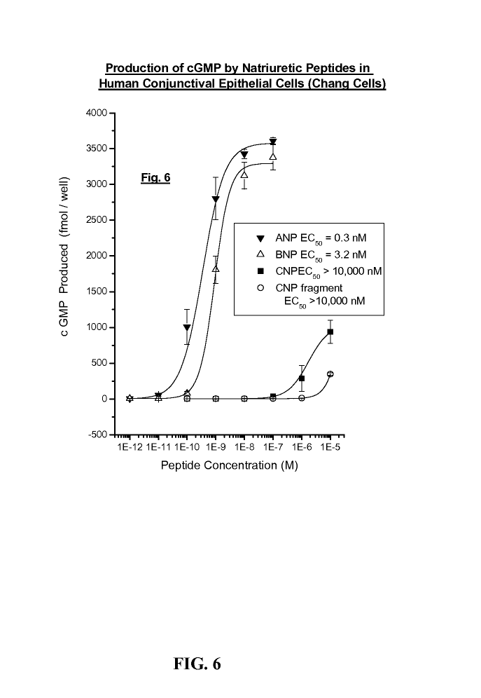

The present inventors have discovered the presence of functionally coupled receptors for CNP in human corneal epithelial cells where CNP potently stimulates cGMP production (EC₅₀=7.0±2.6 nM (n=7)) with ANP being less potent (EC₅₀=97±25 nM; n=4) (see FIGS. 1 and 2; Table 1 below). ANP was a very low efficacy partial agonist relative to CNP indicating the presence of type-B receptors in these cells. The CNP fragment-induced responses were concentration-dependently blocked by a type-B natriuretic peptide receptor antagonist, HS-142-1 in primary human corneal epithelial cells (Kᵢ=458±36 nM, n=6; FIG. 3) and in immortalized human corneal epithelial (CEPI-17-CL4) cells (Kᵢ=208±63 nM, n=6; FIG. 4), but somewhat weakly by another antagonist, isatin (Kᵢ=18±4 μM, in CEPI-17-CL4 cells, n=3; FIG. 5). These latter antagonists are non-peptidic organic molecules and represent agents that could be readily formulated in a suitable fashion to block the effects of CNP/ANP in vivo to render them effective to treat edematous conditions. In contrast, type-A receptors activated by ANP were found in human conjunctival epithelial cells (Chang cells) where ANP and BNP were potent full agonists (ANP EC₅₀=0.34±0.16 nM, n=6; BNP EC₅₀=3.2±2.2 nM, n=4) but where CNP was a weak partial agonist (Table 2; FIG. 6). ANP and CNP synthesis, and CNP-binding sites, occur in the bovine corneal endothelium (Walkenbach et al. Atrial natriuretic peptide receptors on the corneal endothelium. Invest. Opthalmol. Vis. Sci. 34: 2538-2543, 1993; Sung et al., Coexistence of C-type natriuretic peptide and atrial natriuretic peptide systems in the bovine cornea. Invest. Opthalmol. Vis. Sci. 41: 2671-2677, 2000).

It is believed that over-stimulation of the B-type receptor, either due to an excessive release of the natriuretic peptides or when the receptors are pathologically (and perhaps constitutively) active, could cause an excessive amount of fluid secretion and fluid accumulation resulting in edema. If such pathology occurs in the cornea, conjunctiva or in the retina, sight threatening consequences could ensue. Likewise, edema in the inner ear could have deleterious effects on hearing. Therefore, administration of suitably formulated CNP/ANP antagonists would prove to be therapeutically useful for the eye and ear. Such CNP/ANP antagonists are available in limited numbers and none have been turned into drugs yet. However, isatin and its analogs (Medvedev et al. Efficacy of isatin analogues as antagonists of rat brain heart natriuretic peptide receptors coupled to particulate guanylyl cyclase. Biochem. Pharmacol. 57: 913-915, 1999), and HS-142-2 (Matsuda, Y. Design and utilization of natriuretic peptide antagonists. In: Contemporary Endocrinology: Natriuretic Peptides in Health and Disease. (Samson, W. K. and Levin, E. R.; Editors), Humana Press Inc., Totowa, N.J., 1997) (relatively small molecules; non-peptidic in nature) and their isomers and derivatives are expected to be useful in the methods of the present invention. Kambayashi and colleagues (Kambayashi et al. A dicarba analog of beta-atrial natriuretic peptide (beta-ANP) inhibits guanosine-3′,5′-monphosphate production induced by alpha-ANP in cultured rat vascular smooth muscle cells. FEBS. Lett. 248: 28-34, 1989) have described a peptide antagonist for the type-A receptor [Asu-7,23′]b-ANP-(7-28)], while a monoclonal antibody (3G12) appears to behave as an antagonist at the type-B receptor (Drewett et al. Natriuretic peptide receptor-B (guanylyl cyclase-B) mediates C-type natriuretic peptide relaxation of precontracted rat aorta. J. Biol. Chem. 270: 4668-4674, 1995). Other peptide antagonists for natriuretic peptide receptors have also been reported, including anantin (Weber et al. Anantin—a peptide antagonist of the atrial natriuretic factor (ANF). I. Producing organism, fermentation, isolation and biological activity. J. Antibiotics, 44: 164-171, 1991; Abell et al. Competitive peptide antagonists of ANF-induced cyclic guanosine monophosphate production. Biochem. Biophys. Res. Comma 164: 108-113, 1989; Von Geldem et al. Atrial natriuretic peptide antagonists: biological evaluation and structural correlations. Mol. Pharmacol. 38: 771-778, 1990).

Generally, the compounds for use in the methods of the present invention may be administered by any known, acceptable method of delivery for ocular, otic or nasal administration. For ocular administration, preferred methods of delivery include, but are not limited to, intravitreal, topical ocular, transdermal patch, subdermal, parenteral, intraocular, subconjunctival, or retrobulbar or subtenon's injection, trans scleral (including iontophoresis), or slow release biodegradable polymers or liposomes may require an adjustment of the total daily dose necessary to provide a therapeutically effective amount of the compound. The Compounds can also be delivered in ocular irrigating solutions. Concentrations should range from about 0.001 μM to about 100 μM, is preferably about 0.01 μM to about 50 μM.

As stated above, the Compounds can be incorporated into various types of ophthalmic formulations for delivery to the eye (e.g., topically, intracamerally, intravitreal, or via an implant). They may be combined with opthalmologically acceptable preservatives, surfactants, viscosity enhancers, gelling agents, penetration enhancers, buffers, sodium chloride, and water to form aqueous, sterile ophthalmic suspensions or solutions or preformed gels or gels formed in situ. Ophthalmic solution formulations may be prepared by dissolving the compound in a physiologically acceptable isotonic aqueous buffer. Further, the ophthalmic solution may include an opthalmologically acceptable surfactant to assist in dissolving the compound. The ophthalmic solutions may contain a viscosity enhancer, such as, hydroxymethylcellulose, hydroxyethylcellulose, hydroxypropylmethylcellulose, methylcellulose, polyvinyl-pyrrolidone, or the like, to improve the retention of the formulation in the conjunctival sac. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-940, or the like, according to the published formulations for analogous ophthalmic preparations; preservatives and tonicity agents can be incorporated.

If dosed topically, the Compounds are preferably formulated as topical ophthalmic suspensions or solutions, with a pH of about 4 to 8. The Compounds will normally be contained in these formulations in an amount 0.001% to 5% by weight, but preferably in an amount of 0.01% to 2% by weight. Thus, for topical presentation, 1 to 2 drops of these formulations would be delivered to the surface of the eye 1 to 4 times per day according to the discretion of a skilled clinician.

Suitably formulated Compounds (suspensions or solutions; pH 4-8) could be administered to the ear canal for otic utility and applied as drops or as a spray into either or both nostrils for nasal delivery. The Compounds will normally be contained in these formulations in an amount 0.001% to 5% by weight, but preferably in an amount of 0.01% to 2% by weight.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### In Vitro Assays to Discover Natriuretic Peptide Receptor Antagonists

The following methods can be utilized by those skilled in the art to assess the agonist and antagonist activity at type-A and type-B natriuretic peptide receptors. Agonists are identified when they stimulate the production of cGMP. Antagonists would be inactive as stimulators of cGMP production but would instead block the activity of the agonists. In some cases, partial agonists of very low intrinsic activity (Eₘₐₓ) behave as antagonists (e.g. see U.S. Pat. Nos. 6,441,033; and 6,492,417; Griffin et al. AL-8810: a novel PGF_{2α} analog with selective antagonist effects at the FP prostaglandin receptor. J. Pharmacol. Expt. Ther. 290: 1278-1284, 1999; Sharif et al. AL-3138 antagonizes FP prostanoid receptor-mediated inositol phosphates generation: comparison with some purported FP antagonists. J. Pharmac. Pharmacol. 52: 1529-1539, 2000) and thus very low efficacy partial agonists/analogs of ANP or CNP would be expected to antagonize activity at type-A and type-B natriuretic peptide receptors.

Tissue Culture

In order to discover agonists and antagonists for the type-B natriuretic peptide receptor the most relevant cells are human corneal epithelial cells. Thus, primary human corneal epithelial cells (P-CEPI) were isolated from human donor eyes ≦24 hr postmortem and were cultured in EpiLife medium (Cascade Biologics, Portland, Oreg.) containing 1% human corneal growth supplement, 100 u/ml penicillin G, 100 mg/ml streptomycin sulfate. Culture plates were coated with fibronectin to help the cells adhere to the bottom of the plates. Immortalized human corneal epithelial cells (CEPI-17-CL4; U.S. Pat. No. 6,284,537; Sharif et al. Human corneal epithelial cell functional response to inflammatory agents and their antagonists. Invest. Opthalmol. Vis. Sci. 39: 2562-2571, 1998; Offord et al. Immortalized human corneal epithelial cells for ocular toxicity and inflammation studies. Invest. Opthalmol. Vis. Sci. 40: 1091-1101, 1999) (passages 58-158)) were cultured in keratinocyte growth medium (KGM) with 0.15 mM CaCl₂. Amphotericin B and gentamycin were replaced by penicillin (100 U/ml) and streptomycin (100 mg/ml). Media and other supplements were purchased from Cambrex Bio Science Walkersville, Inc. (Walkersville, Md.). The morphologic, pharmacological, and genetic characterization of the simian virus 40-immortalized human corneal epithelial cells (CEPI-17-CL4), that faithfully represent normal primary cells, has been previously reported. (Sharif et al. Human corneal epithelial cell functional response to inflammatory agents and their antagonists. Invest. Opthalmol. Vis. Sci. 39: 2562-2571, 1998; Offord et al. Immortalized human corneal epithelial cells for ocular toxicity and inflammation studies. Invest. Opthalmol. Vis. Sci. 40: 1091-1101, 1999; U.S. Pat. No. 6,284,537).

In order to discover agonists and antagonists for the type-A natriuretic peptide receptor, the most relevant cells are Chang cells (Clone 1-5C-4 Wong-Kilbourne Derivative). These cells were obtained from ATCC (Manassas, VI) and cultured in medium 199, containing 10% fetal bovine serum and 50 mg/ml Gentamicin. (obtained from Gibco/Invitrogen; Carlsbad, Calif.).

Measuring Activity of Guanylyl Cyclase Activity in Cultured Cells

Activity of guanylyl cyclase was measured by the peptide-induced accumulation of cGMP as previously described with some significant modifications (Zhou et al. Multiple cyclic nucleotide phosphodiesterases in human trabecular meshwork cells. Invest. Opthalmol. Vis. Sci. 40: 1745-1752, 1999; Medevedev et al. Effects of isatin on atrial natriuretic peptide-mediated accumulation of cGMP and guanylyl cyclase activity of PC12 cells. Life Sci. 69: 1783-1790, 2001). In brief, compounds of interest were diluted in ethanol so that the final ethanol concentration was 1%, a concentration well tolerated by the cells. Cells were seeded in 48-wells culture plates. On reaching confluence, the cells were rinsed twice with 0.5 ml Dulbeco's modified Eagle's medium (DMEM)/F-12. The cells were pre-incubated for 20 minutes in the presence or absence of either atrial natriuretic peptide (ANP) or natriuretic peptide (CNP) receptor antagonists (isatin or HS-142-1) in DMEM/F-12 containing 1.0 mM of the phosphodiesterase inhibitor 3-isobutyl-1-methylxanthine (IBMX;Sigma-Aldrich, St. Louis, Mo.) at 23° C. Natriuretic peptides (American Peptide Co. Inc., Sunnyvale, Calif.; Peninsula Labs. San Carlos, Calif.) were then added at the end of this period and the reaction was allowed to proceed for another 15 minutes at 23° C. After aspiration of the reaction medium, ice cold 0.1 M acetic acid (150 ml, pH 3.5) was added for the termination of cGMP synthesis and cell lysis. Finally, ice-cold 0.1 M sodium acetate (220 ml, pH 11.5-12.0) was added to neutralize the samples before analysis of cGMP by an enzyme immunoassay (EIA) kit. cGMP production in the cells was measured using the EIA kit purchased from Amersham Pharmacia Biotech (Piscataway, N.J.). This assay was conducted according to the package insert in an automated manner using a robotic workstation (Biomek 2000; Beckman Instrument, Fullerton, Calif.) (Sharif et al. Characterization of the ocular anti-allergic and anti-histaminic effects of Olopatadine (AL-4943A), a novel drug for treating ocular allergic diseases. J. Pharmacology & Experimental Therapeutics 278, 1251-1260, 1996; Crider et al. Pharmacological characterization of serotonin receptor (5HT₇) stimulating cAMP production in human corneal epithelial cells. Invest. Opthalmol. Vis. Sci. 44: 4837-4844, 2003). Data were analyzed using a non-linear, iterative curve-fitting computer program (IDBS, Surrey, UK) and Origin software package (Microcal Software, Inc., Northampton, Mass.) (Sharif et al. 1996; Crider et al. 2003). The natriuretic peptide agonist potencies (EC₅₀; concentration required to achieve 50% of the maximum activity) and the antagonist potencies (Kᵢ; concentration required to achieve 50% inhibition of the maximum agonist-induced activity) were determined from multiple experiments to obtain the means±SEMs of the data. Typical concentration-response curves for natriuretic peptides in normal primary human corneal epithelial (FIG. 1) and in CEPI-17-CL4 cells (FIG. 2) are shown. Antagonist activity of HS-142-1 against CNP-fragment in these cells is shown in FIGS. 3 and 4, while antagonist data for isatin are shown in FIG. 5. Data from several experiments for the agonist agents have been described above and are shown in Table 1. Agonist data for type-A natriuretic peptide receptors obtained from Chang cells are shown in FIG. 6 and Table 2.

TABLE 1
Agonist Potencies of Natriuretic Peptides Stimulating cGMP Production
via Type-B Receptor in Human Corneal Epithelial Cells

| | | | Intrinsic Activity |
|---|---|---|---|
| Natriuretic | | Potency | (Eₘₐₓ; % Max. |
| Peptide | Species | EC₅₀ (nM) | Response) |
| CNP | Human | 7.0 ± 2.6 | 100% |
| (1-53 amino | | (n = 7) | |
| acids) | | | |
| CNP Fragment | Human | 21.0 ± 4.7 | 100% |
| (32-53 amino | | (n = 6) | |
| acids) | | | |
| BNP | Human | 54.0 ± 4.0 | 50.2% |
| (32 amino | | (n = 2) | |
| acids) | | | |
| ANP | Human | 97.0 ± 25.0 | 10.8% |
| (1-28 amino | | (n = 4) | |
| acids) | | | |

TABLE 2
Agonist Potencies of Natriuretic Peptides Stimulating cGMP Production
via Type-A Receptor in Human Conjunctival Epithelial Cells
(Chang Cells)

| | | | Intrinsic Activity |
|---|---|---|---|
| Natriuretic | | Potency | (Eₘₐₓ; % Max. |
| Peptide | Species | EC₅₀ (nM) | Response) |
| CNP | Human | >10,000 | 26% |
| (1-53 amino | | | |
| acids) | | | |
| CNP Fragment | Human | >10,000 | 10% |
| (32-53 amino | | | |
| acids) | | | |
| BNP | Human | 3.2 ± 2.2 | 94% |
| (32 amino | | (n = 4) | |
| acids) | | | |
| ANP | Human | 0.34 ± 0.16 nM | 100% |
| (1-28 amino | | 0.35 (n = 6) | |
| acids) | | | |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, and all references mentioned herein, to the extent that they provide exemplary procedural or other details supplementary to those set forth herein, are specifically incorporated herein by reference.
Abell et al., Competitive peptide antagonists of ANF-induced cyclic guanosine monophosphate production. Biochem. Biophys. Res. Comm. 164: 108-113, 1989;
Anand-Srivastava and Trachte, Atrial natriuretic factor receptors and signal transduction mechanisms. Pharmacol. Rev. 45: 455-497, 1993.
Crider et al. Pharmacological characterization of serotonin receptor (5HT₇) stimulating cAMP production in human corneal epithelial cells. Invest. Opthalmol. Vis. Sci. 44: 4837-4844, 2003.
Denninger, J W and Marletta, M A. Guanylate cyclase and the NO/cGMP signaling pathway. Biochemica et Biophysica Acta 1411: 334-350, 1999.
Drewett et al., Natriuretic peptide receptor-B (guanylyl cyclase-B) mediates C-type natriuretic peptide relaxation of precontracted rat aorta. J. Biol. Chem. 270: 4668-4674, 1995.
Griffin et al., AL-8810: a novel PGF_{2α} analog with selective antagonist effects at the FP prostaglandin receptor. J. Pharmacol. Expt. Ther. 290: 1278-1284, 1999.
Kambayashi et al., A dicarba analog of beta-atrial natriuretic peptide (beta-ANP) inhibits guanosine-3′,5′-monphosphate production induced by alpha-ANP in cultured rat vascular smooth muscle cells. FEBS. Lett. 248: 28-34, 1989
Lange et al., Localization of atrial natriuretic peptide/cariodilatin (ANP/CDD)-immunoreactivity in the lacrimal gland of the domestic pig. Exp. Eye Res. 50: 313-316, 1990.
Matsuda, Y and Morishita, Y. HS-142-1: a novel nonpeptide atrial natriuretic peptide antagonist of microbial origin. Cardiovasc. Drug Rev. 11: 45-59, 1993.
Matsuda, Y, Design and utilization of natriuretic peptide antagonists. In: Contemporary Endocrinology: Natriuretic Peptides in Health and Disease. (Samson, W. K. and Levin, E. R.; Editors), Humana Press Inc., Totowa, N.J. (1997)
Medvedev et al., Isatin: a link between natriuretic peptides and monoamines? Biochem. Pharmacol. 52: 385-399, 1996.
Medvedev et al., Interaction of isatin with type-A natriuretic peptide receptor: possible mechanism. Life Sci. 62: 2391-2398, 1998.
Medvedev et al., Efficacy of isatin analogues as antagonists of rat brain heart natriuretic peptide receptors coupled to particulate guanylyl cyclase. Biochem. Pharmacol. 57: 913-915, 1999.
Medevedev et al., Effects of isatin on atrial natriuretic peptide-mediated accumulation of cGMP and guanylyl cyclase activity of PC12 cells. Life Sci. 69: 1783-1790, 2001.
Offord et al., Immortalized human corneal epithelial cells for ocular toxicity and inflammation studies. Invest. Opthalmol. Vis. Sci. 40: 1091-1101, 1999.
Sharif et al., Characterization of the ocular anti-allergic and anti-histaminic effects of Olopatadine (AL-4943A), a novel drug for treating ocular allergic diseases. J. Pharmacology & Experimental Therapeutics 278, 1251-1260, 1996.
Sharif et al., Human corneal epithelial cell functional response to inflammatory agents and their antagonists. Invest. Opthalmol. Vis. Sci. 39: 2562-2571, 1998.
Sharif et al., AL-3138 antagonizes FP prostanoid receptor-mediated inositol phosphates generation: comparison with some purported FP antagonists. J. Pharmac. Pharmacol. 52: 1529-1539, 2000.
Sung et al., Coexistence of C-type natriuretic peptide and atrial natriuretic peptide systems in the bovine cornea. Invest. Opthalmol. Vis. Sci. 41: 2671-2677, 2000.
Von Geldern et al., Atrial natriuretic peptide antagonists: biological evaluation and structural correlations. Mol. Pharmacol. 38: 771-778, 1990.
Walkenbach et al., Atrial natriuretic peptide receptors on the corneal endothelium. Invest. Opthalmol. Vis. Sci. 34: 2538-2543, 1993.
Weber et al., Anantin—a peptide antagonist of the atrial natriuretic factor (ANF). I. Producing organism, fermentation, isolation and biological activity. J. Antibiotics, 44: 164-171, 1991.
Yamamoto et al., Brain natriuretic peptide-immunoreactive nerves in the porcine eye. Neurosci. Lett. 122: 151-151, 1991.
Zhang et al., Effects of atrial natriuretic peptide and sodium nitroprusside on epidermal growth factor-stimulated wound repair in rabbit corneal epithelial cells. Curr. Eye Res. 21: 748-756, 2001.
Zhou et al., Multiple cyclic nucleotide phosphodiesterases in human trabecular meshwork cells. Invest. Opthalmol. Vis. Sci. 40: 1745-1752, 1999.

### U.S. Pat. Nos.

6,284,537
6,441,033,
6,492,417
6,531,480

We claim:

1. A method for treating an ocular edematous disorder, said method comprising administering a composition comprising a therapeutically effective amount of a non-peptide or stabilized peptide natriuretic peptide (ANP/BNP/CNP) receptor antagonist.

2. The method of claim 1, wherein the ocular edematous disorder is selected from the group consisting of corneal, conjunctival edema, retinal edema, and retinal detachment.

3. The method of claim 1, wherein the natriuretic peptide receptor antagonist is a peptide or non-peptide ANP/BNP/CNP analog or mimetic that exhibits antagonist activity at type-A and/or type-B natriuretic peptide receptors.

4. The method of claim 3, wherein the natriuretic peptide receptor antagonist is selected from the group consisting of HS-142-1, isatin, [Asu-7,23′]b-ANP-(7-28)], anantin, 3G12, and analogs thereof.

5. The method of claim 4, wherein the natriuretic peptide receptor antagonist is isatin.

6. The method of claim 4, wherein the natriuretic peptide receptor antagonist is HS-142-1.

7. The method of claim 4, wherein the natriuretic peptide receptor antagonist is an analog or derivative of isatin or HS-142-1.

8. The method of claim 1, wherein the amount of the natriuretic peptide receptor antagonist in the composition is from 0.01% to 2% by weight.

9. The method of claim 1, wherein the concentration of the natriuretic peptide receptor antagonist is from about 0.01 μM to about 50 μM.

10. A method for treating an otic edematous disorder, said method comprising administering a composition comprising a therapeutically effective amount of a non-peptide or stabilized peptide natriuretic peptide receptor antagonist.

11. The method of claim 10, wherein the natriuretic peptide receptor antagonist is a peptide or non-peptide ANP/BNP/CNP analog or mimetic that exhibits antagonist activity at type-A and/or type-B natriuretic peptide receptors.

12. The method of claim 11, wherein the natriuretic peptide receptor antagonist is selected from the group consisting of HS-142-1, isatin, [Asu-7,23′]b-ANP-(7-28)], anantin, 3G12, and analogs thereof.

13. The method of claim 12, wherein the natriuretic peptide receptor antagonist is isatin.

14. The method of claim 12, wherein the natriuretic peptide receptor antagonist is HS-142-1.

15. The method of claim 10, wherein the amount of natriuretic peptide receptor antagonist in the composition is from 0.01% to 2% by weight.

16. The method of claim 10, wherein the concentration of natriuretic peptide receptor antagonist receptor in the composition is from about 0.01 μM to about 50 μM.

17. A method for treating a nasal edematous disorder, said method comprising administering a composition comprising a therapeutically effective amount of a non-peptide or stabilized peptide natriuretic peptide receptor antagonist.

18. The method of claim 17, wherein the natriuretic peptide receptor antagonist is a peptide or non-peptide ANP/BNP/CNP analog or mimetic that exhibits antagonist activity at type-A and/or type-B natriuretic peptide receptors.

19. The method of claim 18, wherein the natriuretic peptide receptor antagonist is selected from the group consisting of HS-142-1, isatin, [Asu-7,23′]b-ANP-(7-28)], anantin, 3G12, and analogs thereof.

20. The method of claim 19, wherein the natriuretic peptide receptor antagonist is isatin.

21. The method of claim 19, wherein the natriuretic peptide receptor antagonist is HS-142-1.

22. The method of claim 17, wherein the amount of natriuretic peptide receptor antagonist in the composition is from 0.01% to 2% by weight.

23. The method of claim 17, wherein the concentrations of natriuretic peptide receptor antagonist in the composition is from about 0.01 μM to about 50 μM.
